(19) United States
(12) Patent Application Publication
(10) Pub. No.: US 2025/0275725 A1
(43) Pub. Date: Sep. 4, 2025

(54) ECG DATA COMPRESSION AND DECOMPRESSION

(71) Applicant: Boston Scientific Cardiac Diagnostics, Inc., Rochester, MN (US)

(72) Inventors: Michael Thomas Edward McRoberts, Mazeppa, MN (US); Jake Matras, Eagan, MN (US)

(21) Appl. No.: 19/059,607

(22) Filed: Feb. 21, 2025

Related U.S. Application Data
(60) Provisional application No. 63/560,996, filed on Mar. 4, 2024.

(51) Int. Cl. A61B 5/00; A61B 5/346

(52) U.S. Cl. CPC A61B 5/7232; A61B 5/0006; A61B 5/346; A61B 5/7267

(57) ABSTRACT

Systems, methods, and devices utilize approaches for compressing electrocardiogram (ECG) data or other time-series data. Approaches involve processing strips of ECG data to generate compressed packages of the ECG data. The processing includes generating preconditioned ECG data and compressing the preconditioned ECG data. The compressed packages of ECG data can be transmitted to a remote computing system.

### CROSS REFERENCE TO RELATED APPLICATION

This application claims priority to Provisional Application No. 63/560,996, filed Mar. 4, 2024, which is herein incorporated by reference in its entirety.

### TECHNICAL FIELD

The present disclosure relates to devices, methods, and systems for compressing time series data such as electrocardiogram data.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Cardiac monitoring can be used, for example, to identify abnormal cardiac rhythms, so that alerts can be provided to patients, physicians, or other care providers and patients can be treated.

### SUMMARY

In Example 1, a method includes receiving one or more electronic files that contain strips of electrocardiogram (ECG) data, processing the strips of the ECG data to generate compressed packages of the ECG data, and transmitting the compressed packages of the ECG data to a remote computing system. The processing includes generating preconditioned ECG data and compressing the preconditioned ECG data.
In Example 2, the method of Example 1, wherein the generating the preconditioned ECG data includes processing the strips of ECG data using a first encoding process to generate a first set of encoded ECG data, and then processing the first set of encoded ECG data to generate a second set of encoded ECG data, wherein the second set of encoded data is the preconditioned ECG data.
In Example 3, the method of Example 1, wherein the generating the preconditioned ECG data includes reducing bias in the ECG data, wherein the compressing the preconditioned ECG data includes using bit packing.
In Example 4, the method of Example 3, wherein, after the reducing the bias, the ECG data contains initial negative values and initial positive values.
In Example 5, the method of Example 4, wherein the generating the preconditioned ECG data further includes: converting the initial negative values to subsequent positive values.
In Example 6, the method of Example 5, wherein the subsequent positive values are interleaved with the initial positive values to generate the preconditioned ECG data.
In Example 7, the method of any of Examples 3-6, wherein the reducing the bias in the ECG data includes applying a differential encoding algorithm to the ECG data.
In Example 8, the method of any of Examples 1-7, wherein the compressed packages of data include 128-bit-sized arrays.
In Example 9, the method of any of Examples 1-8, wherein each of the strips is separately processed to generate a separate compressed package.
In Example 10, the method of any of Examples 1-9, further including: before the processing the strips of the ECG data to generate the compressed packages of the ECG data, inputting the strips of the ECG data into a machine learning model to generate metadata, the metadata including cardiac event classifications and beat classifications.
In Example 11, the method of any of Examples 1-10, wherein the compressed packages of the ECG data include a collection of separate blocks of compressed strips of ECG data.
In Example 12, the method of any of Examples 1-11, further including: transmitting executable code to the remote computing system, wherein the executable code includes instructions to be executed by the remote computing system to decompress the compressed packages of the ECG data.
In Example 13, a computer program product comprising instructions to cause one or more processors to carry out the steps of the method of Examples 1-12.
In Example 14, a computer-readable medium having stored thereon the computer program product of Example 13.
In Example 15, a server comprising the computer-readable medium of Example 14.
In Example 16, a system includes a server with one or more processors and computer-readable media. The media having computer-executable instructions embodied thereon. The instructions are configured to be executed by the one or more processors to cause the server to: process strips of ECG data to generate compressed packages of the ECG data and transmit the compressed packages of the ECG data to a remote computing system. The processing includes generating preconditioned ECG data and compressing the preconditioned ECG data.
In Example 17, the system of Example 16, wherein the generating the preconditioned ECG data includes processing the strips of ECG data using a first encoding process to generate a first set of encoded ECG data, and then processing the first set of encoded ECG data to generate a second set of encoded ECG data, wherein the second set of encoded data is the preconditioned ECG data.
In Example 18, the system of Example 16, wherein the generating the preconditioned ECG data includes reducing bias in the ECG data, wherein the compressing the preconditioned ECG data includes using bit packing.
In Example 19, the system of Example 18, wherein, after the reducing the bias, the ECG data contains initial negative values and initial positive values.
In Example 20, the system of Example 19, wherein the generating the preconditioned ECG data further includes: converting the initial negative values to subsequent positive values.
In Example 21, the system of Example 20, wherein the subsequent positive values are interleaved with the initial positive values to generate the preconditioned ECG data.
In Example 22, the system of Example 18, wherein the reducing the bias in the ECG data includes applying a differential encoding algorithm to the ECG data.
In Example 23, the system of Example 16, wherein the server comprises a machine learning mode. The instructions are configured to be executed by the one or more processors to cause the server to input the strips of the ECG data into the machine learning model to generate metadata, which includes cardiac event classifications and beat classifications. In certain instances, inputting the strips into the machine learning model is carried out before the strips of the ECG data are processed to generate the compressed packages of the ECG data.
In Example 24, the system of Example 16, wherein the compressed packages of the ECG data include a collection of separate blocks of compressed strips of ECG data.
In Example 25, the system of Example 16, wherein the compressed packages of the ECG data that contain a critical cardiac event are transmitted to the remote computing system before the compressed packages of ECG data that do not contain a critical cardiac event.
In Example 26, the system of Example 16, wherein the instructions are configured to be executed by the one or more processors to cause the server to: transmit executable code to the remote computing system. The executable code is configured to be executed by the remote computing system to decompress the compressed packages of the ECG data.
In Example 27, the system of Example 26, further including the remote computing system with a set of its own one or more processors. The one or more processors are arranged to execute the executable code to cause the remote computing system to: decompress the compressed packages of the ECG data into the preconditioned ECG data decode the preconditioned ECG data to reconstruct the strips of the ECG data.
In Example 28, the system of Example 27, wherein the remote computing system includes a user interface, wherein the user interface is configured to display the strips ECG data after being decompressed and decoded.
In Example 29, the system of Example 27, wherein the remote computing system is configured to operate a browser, wherein the browser is configured to execute the executable code received from the server.
In Example 30, a method includes receiving one or more electronic files that contain strips of time-series data, processing the strips of the time-series data to generate compressed packages of the time-series data, and transmitting the compressed packages of the time-series data to a remote computing system. The processing includes generating preconditioned time-series data and compressing the preconditioned time-series data. Generating the preconditioned time-series data includes using a first encoding process to convert the time-series data to a first set of encoded time-series data and then using a second encoding process to convert the first set of encoded time-series data to the preconditioned time-series data.
In Example 31, the method of Example 30, wherein the first set of encoded time-series data includes initial negative values and initial positive values.
In Example 32, the method of Example 31, wherein the second set of encoded time-series data only includes positive values.
In Example 33, the method of Example 32, wherein the positive values are compressed using bit packing.
In Example 34, the method of Example 32, wherein the positive values include subsequent positive values converted from the initial negative values, wherein the subsequent positive values are interleaved with the initial positive values to generate the preconditioned time-series data.
In Example 35, the method of Example 30, wherein the first encoding process applies a differential encoding algorithm to the time-series data.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, remote computer, and user interface, in accordance with certain instances of the present disclosure.

FIG. 3 shows a flow diagram depicting an illustrative method for compressing ECG data, in accordance with instances of the disclosure.

FIG. 4 shows a flow diagram depicting an illustrative subroutine of the method of FIG. 3, in accordance with instances of the disclosure.

FIG. 5 shows a diagram of data structures of compressed ECG data, in accordance with instances of the disclosure.

FIG. 6 shows a diagram of an index of compressed ECG data, in accordance with instances of the disclosure.

FIG. 7 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

The present disclosure relates to devices, methods, and systems that compress electrocardiogram (ECG) data and/or utilize compressed ECG data. Processing and transmitting uncompressed ECG data can consume a large amount of computing resources and network bandwidth resources. Instances of the present disclosure are accordingly directed to approaches for facilitating compression (and decompression) of ECG data.

Before describing particular ECG data compression and decompression approaches, the description below outlines an example system in which compressed ECG data can be used. The description also describes ways ECG data can be analyzed to determine whether a patient has experienced abnormal cardiac activity.

Although the examples herein are focused on compressing ECG data, other types of time-series data could be compressed using the techniques described herein.

### System

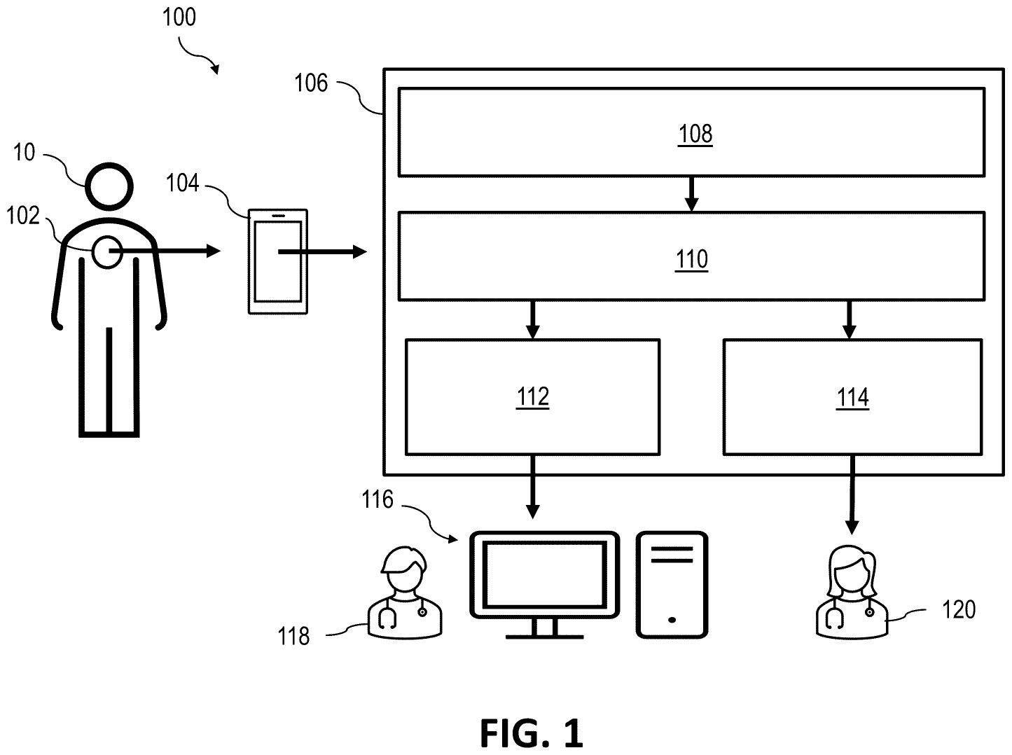

FIG. 1 illustrates a patient 10 and an example system 100. The system 100 includes a monitor 102 attached to the patient 10 to detect cardiac activity of the patient 10. The monitor 102 may produce electric signals that represent the cardiac activity in the patient 10. For example, the monitor 102 may detect the patient's heart beating (e.g., using infrared sensors, electrodes) and convert the detected heartbeat into electric signals representing ECG data. The monitor 102 communicates the ECG data to a mobile device 104 (e.g., a mobile phone).

The mobile device 104 may include a program (e.g., mobile phone application) that receives, processes, and analyzes the ECG data. For example, the program may analyze the ECG data and detect or flag cardiac events (e.g., periods of irregular cardiac activity) contained within the ECG data. Because ECG data may be getting continuously generated, the amount of ECG data can be overwhelming to store and process locally on the mobile device 104. As such, the mobile device 104 can periodically transmit chunks of the ECG data to another device or system, which can process, append together, and archive the chunks of the ECG data and metadata (e.g., time, duration, detected/flagged cardiac events) associated with the chunks of ECG data. In certain instances, the mobile device 104 is programmed to compress the ECG data before the ECG data is transmitted to another device or system. This can reduce the amount of memory that is used or required to store ECG data and can reduce the amount of networking resources used or required to transmit ECG data to another device or system.

The monitor 102 may be programmed to transmit the ECG data directly to another device or system without utilizing the mobile device 104. In instances involving multi-day studies, the ECG data and associated metadata can be transmitted in larger chunks.

### Cardiac Event Server

In the example shown in FIG. 1, the mobile device 104 transmits the ECG data (compressed or uncompressed) and associated metadata, if any, to a cardiac event server 106 (hereinafter “the server 106” for brevity). The server 106 includes multiple platforms, layers, or modules that work together to process and analyze the ECG data such that cardiac events can be detected, filtered, prioritized, and ultimately reported to a patient's physician for analysis and treatment. If the ECG data transmitted to the server 106 is compressed, the server 106 can decompress the ECG data before processing and analyzing the ECG data.

In the example of FIG. 1, the server 106 includes one or more machine learning models 108 (e.g., types of deep neural networks), a cardiac event router 110, a report platform 112, and a notification platform 114. Although only one server 106 is shown in FIG. 1, the server 106 can include multiple separate physical servers, and the various platforms/modules/layers can be distributed among the multiple servers. Each of the platforms/modules/layers can represent separate programs, applications, and/or blocks of code where the output of one of the platforms/modules/layers is an input to another of the platforms/modules/layers. Each platform/module/layer can use application programming interfaces to communicate between or among the other platforms/modules/layers as well as systems and devices external to the server 106.

The server 106 applies the machine learning model 108 to the ECG data to classify cardiac activity of the patient 10. For example, the machine learning model 108 may compare the ECG data to labeled ECG data to determine which labeled ECG data the ECG data most closely resembles. The labeled ECG data may identify a particular cardiac event, including but not limited to ventricular tachycardia, bradycardia, atrial fibrillation, pause, normal sinus rhythm, or artifact/noise. Further, the labeled ECG data may identify beat classifications such as normal, ventricular, and supraventricular. For example, the machine learning model 108 can assign each beat with a beat classification. To distinguish among the beats, each individual beat can therefore be assigned a unique identifier (e.g., a unique number).

In certain embodiments, the machine learning model 108 includes two paths, where the first path is a deep convolutional neural network and the second path is a deep fully-connected neural network. The deep convolutional neural network receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, and a non-linear activation function layer. This set of layers can be repeated multiple times.

The deep fully connected neural network can receive RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

If the machine learning model 108 determines that the ECG data most closely resembles a labeled ECG data associated with a cardiac event, then the machine learning model 108 may determine that the patient 10 has experienced that cardiac event. Additionally, the machine learning model 108 may measure or determine certain characteristics of the cardiac activity of the patient 10 based on the ECG data. For example, the machine learning model 108 may determine a heart rate, a duration, or a beat count of the patient 10 during the cardiac event based on the ECG data. The server 106 stores the cardiac event and associated metadata such as information like beat classification, heart rate, duration, beat count, etc., in a database for storage. Subsequently, the server 106 may retrieve the cardiac event and associated information from the database.

In certain instances, the mobile device 104 (or monitor 102) may initially classify a cardiac event based on the ECG data. The server 106 may then re-classify or confirm the cardiac event using the machine learning model 108. Doing so allows for a more computationally-expensive analysis of the ECG data to be performed using the computing resources of the server 106, rather than the limited resources of the mobile device 104.

In certain instances, once the ECG data is processed by the machine learning model 108, the ECG data and metadata is made available for the report platform 112. The report platform 112 can be accessed by a remote computer 116 (e.g., a client device such as a laptop, mobile phone, desktop computer, and the like) by a user at a clinic or lab 118.

In other instances, the cardiac event router 110 is used to determine what platform further processes the ECG data based on the classification associated with the cardiac event. For example, if the identified cardiac event is severe, the cardiac event router 110 can flag or send the ECG data, etc., to the notification platform 114. The notification platform 114 can be programmed to send notifications (along with relevant ECG data and associated metadata) immediately to the patient's physician/care group remote computer and/or to the patient 10 (e.g., to their computer system, e-mail, mobile phone application).

### Report Platform

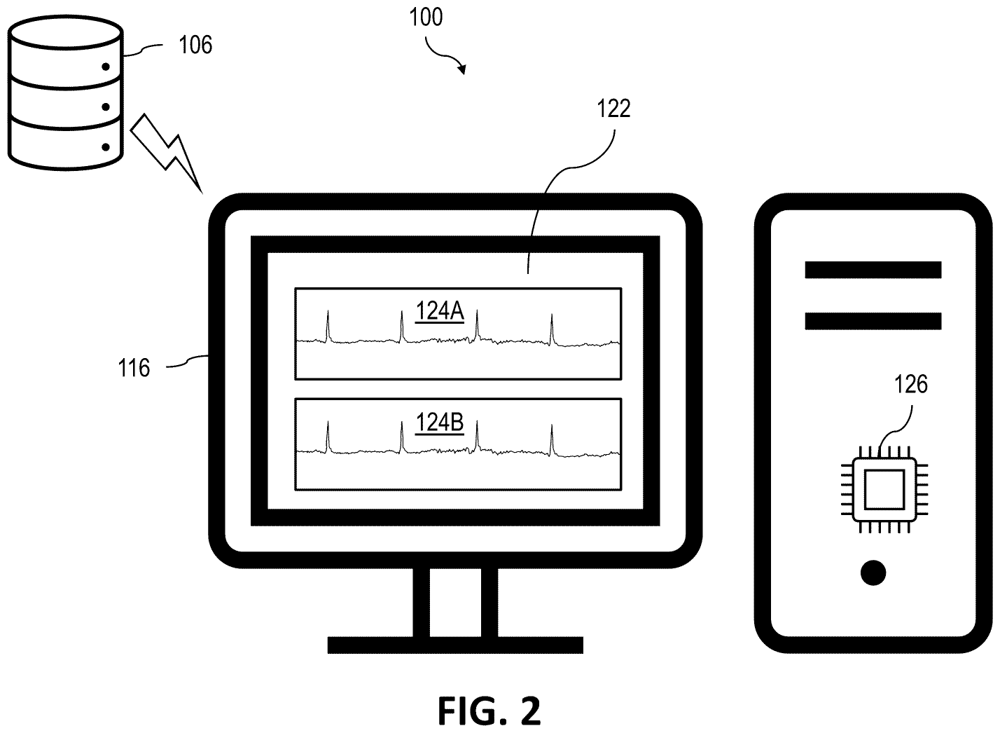

FIG. 2 shows the server 106 communicatively coupled (e.g., via a network) to the remote computer 116. In the example of FIG. 2, the remote computer 116 includes a monitor showing a user interface 122 (hereinafter “the UI 122” for brevity) that displays features of the report platform 112 hosted by the server 106. The UI 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the UI 122 to log into the report platform 112 via a web browser such that the user can use and interact with the report platform 112. When the user at the clinic or lab 118 is ready to analyze ECG data of a patient, the user can select a patient's profile through the UI 122.

The server 106 (e.g., via programming associated with the report platform 112) can start a process for transmitting data to the remote computer 116. This data includes the ECG data and metadata associated with the ECG data.

However, before the ECG data is transmitted to the remote computer 116, the server 106 can compress the ECG data to reduce the amount of network bandwidth resources and computing resources used to transmit and process the ECG data.

### Data Compression and Decompression

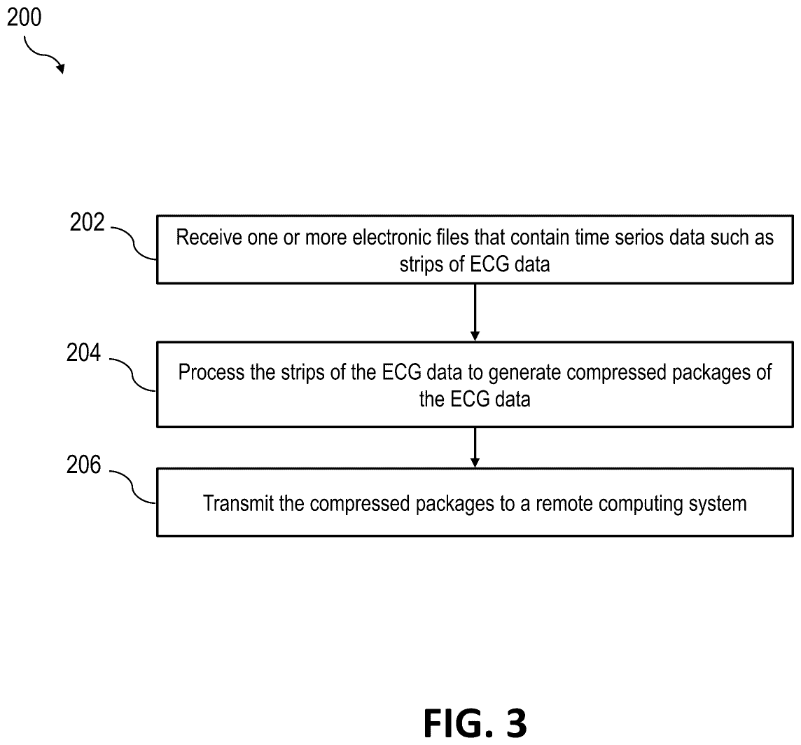

FIG. 3 shows a block diagram of a method 200 for compressing data such as ECG data. The method 200 can be carried out by one or more items within the system 100, including the monitor 102, the mobile device 104 and/or the server 106. For example, the monitor 102 could compress the ECG data before the ECG data is transmitted to the mobile device 104, and the mobile device 104 could compress the ECG data before the ECG data is transmitted to the server 106. The server 106 could then decompress the ECG data, input the uncompressed ECG data into the machine learning model 108, and compress the ECG data again before transmitting the (compressed) ECG data to the remote computer 116, which can decompress the ECG data. In other instances, only the server 106 compresses the ECG data.

The method 200 includes receiving one or more electronic files that contain strips of ECG data (block 202 in FIG. 3). In certain instances, the one or more electronic files are in a text file format such as a .txt file. In certain instances, the mobile device 104 and/or the server 106 receive separate strips of ECG data (e.g., 1-10 minutes strips ECG data such as 6-minute strips) and each strip is stored in separate electronic files. For example, the monitor 102 may be programmed to record the ECG data into separate strips onto local memory and periodically transmit the strips so that new ECG data can be stored to the memory. In other instances, the mobile device 104 and/or the server 106 receive ECG data from an entire study but divide the ECG data into smaller strips of ECG data. For example, the monitor 102 may be programmed and designed to be coupled to the patient 10 for days (e.g., for a multi-day study) and then mailed to a reception center, which downloads days of ECG data, which is split into separate strips of ECG data.

The strips of ECG data can comprise time-series data that reflect sensed cardiac electrical activity of the patient over time (e.g., voltage amplitudes over time). Example visual representations of strips of ECG data are shown in FIG. 2, including a first strip 124A of ECG data and a second strip 124B of ECG data. A multi-day study could include hundreds or thousands of separate strips of ECG data, depending on the length of each strip.

The method 200 further includes processing the strips of the ECG data to generate compressed packages of the ECG data (block 204 in FIG. 3).

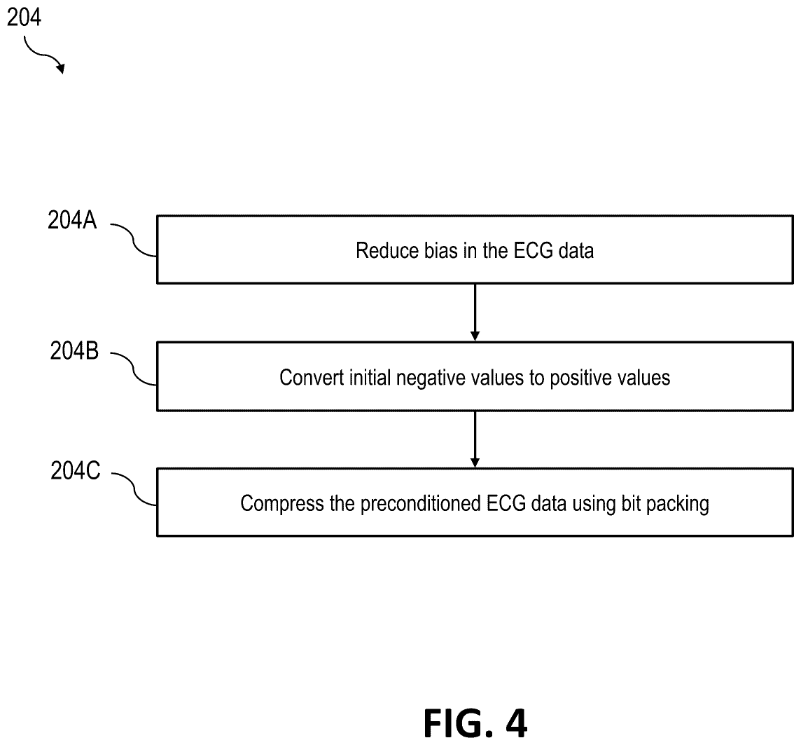

FIG. 4 outlines more detailed steps for generating the compressed packages of ECG data. Before the ECG data is compressed, the ECG data can be preprocessed or preconditioned. Preconditioning ECG data can involve modifying the ECG data such that compression techniques are more effective, e.g., by increasing the compression ratio.

A first preconditioning step can be referred to as a first encoding step, and the first preconditioning step can include reducing bias in the ECG data (block 204A in FIG. 4). As one example, reducing bias can be accomplished by applying a differential encoding algorithm to the ECG data. The differential encoding algorithm can involve calculating a difference between adjacent data points (e.g., a difference in amplitudes of adjacent data points) within the ECG data. Applying a differential encoding algorithm converts the ECG data into a set of either positive values or negative values. Equation 1 below is an example differential encoding equation where dᵢ is the calculated difference between a current data point (eᵢ) and an adjacent data point (eᵢ₋₁):

$${d}_{i} = {e}_{i} - {e}_{i} - 1 \qquad Equation 1$$

After a differential encoding algorithm is applied to the ECG data, the ECG data can be represented by a set of initial negative values and a set of initial positive values.

A second preconditioning step can be referred to as a second encoding step, and the second preconditioning step can include converting the initial negative values to positive values (block 204B in FIG. 4). In addition, the converted initial negative values (e.g., values converted into subsequent positive values) can be interleaved with the initial positive values to generate the preconditioned ECG data. Further, the initial positive values can be shifted or otherwise reordered such that the subsequent positive values can be interleaved with the initial positive values. In certain instances, the process of converting negative values and interleaving is governed by an equation such as Equation 2:

$${z}_{i} = ( {d}_{i} >> bitlength - 1 ) ⊕ ( {d}_{i} << 1 ) \qquad Equation 2$$

where dᵢ is the output of Equation 1 (and the number being encoded), bitlength is the desired bitlength (e.g., 32), “>>” and “<<” are shift operators, and ⊕ is an XOR operator.

Applying Equation 2 results in mapping the initial negative values to subsequent positive values and interleaving the positive values. An example mapping and interleaving pattern is: 0=0, −1=1, 1=2, −2=3, 2=4, −3=5, 3=6, −4=7, 4=8, and so on.

As noted above, in certain instances, the first preconditioning process can be considered a first encoding process, and the second preconditioning process can be considered a second encoding process. The first encoding process can generate a first set of encoded ECG data, which is then an input to the second encoding process that generates a second set of encoded ECG data. The second set of encoded ECG data can be the preconditioned ECG data.

The output of the second preconditioning step can be an input to a compression algorithm. In the example of FIG. 4, the preconditioned ECG data is compressed using bit packing (block 204C in FIG. 4). Bit packing attempts to pack as many bits into a predetermined size data array such as a 128-bit array. Put another way, bit packing approaches attempt to use as few bits as possible to represent data.

A large percentage of ECG data is comprised of time periods with low variability (e.g., the time period between adjacent beats), and these low-variability periods can be represented with a relatively low number of bits. This characteristic of ECG data allows the ECG data to be compressed at a relatively high compression ratio using bit packing approaches. The compression ratio is further improved by using one or more of the preconditioning steps described above.

In certain instances, the strips of ECG data are separately processed to generate separate compressed packages of ECG data. For example, a first strip of ECG data can be preconditioned and then compressed using the approaches described herein —after which a second strip of ECG can be preconditioned and then compressed using the approaches described herein, and so on until all strips have been compressed. As will be described in more detail below, generating separate compressed packages of ECG data allows certain strips of ECG data to be transmitted based on priority and in parallel with each other. However, having hundreds or thousands of separate compressed packages of ECG data for every study of a patient can be challenging to manage, track, transmit, and download.

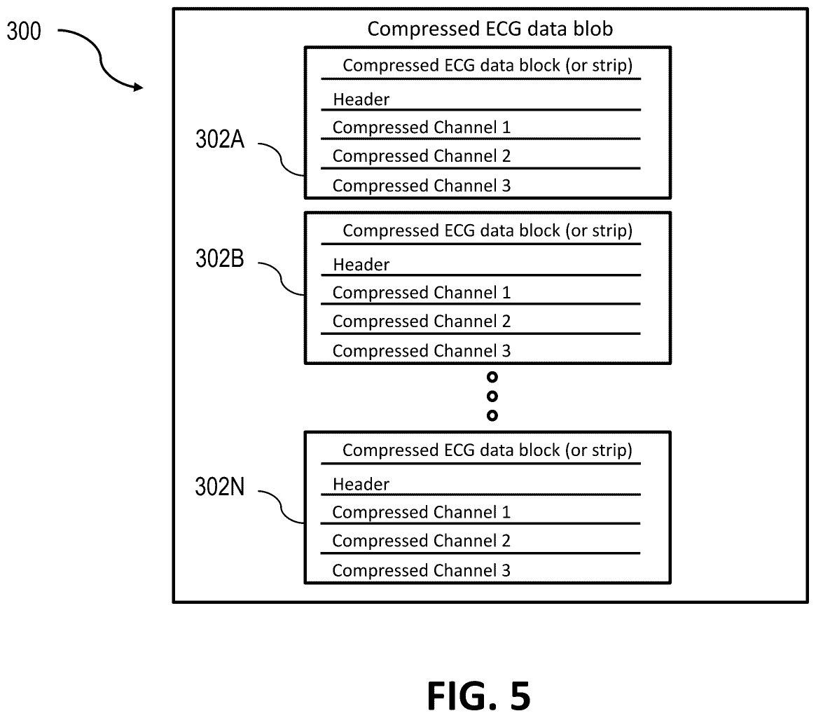

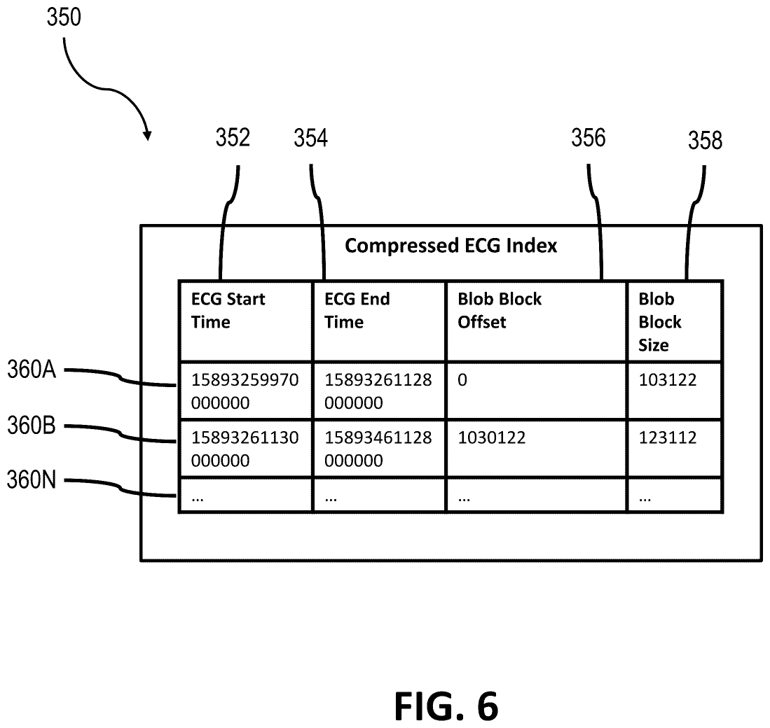

FIGS. 5 and 6 outline data structures and an index that can be useful for managing and tracking separate compressed packages of ECG data—although other data structures and index formats can be used with the compressed packages of ECG data.

FIG. 5 shows a data structure 300 containing compressed ECG data. The data structure 300 may be referred to as a compressed ECG data blob or chunk. The data structure 300 contains an N-number of multiple compressed ECG data blocks or strips 302A-N. When uncompressed, the ECG data blocks 302A-N can represent a contiguous set of ECG data (e.g., adjacent strips of ECG data). The blob block offset value 356 represents the start location in bytes from the start of the “blob” of compressed ECG. This allows a section of compressed ECG to be read from the overall blob of compressed ECG without having to read the entire blob. When uncompressed, the compressed ECG data blocks can be combined (or stitched) together to create a longer, contiguous strip of ECG data.

Each of the compressed ECG data blocks 302A-N can have a header (e.g., with reference information such a start time, end time, index number, and the like) and one or more channels of compressed ECG data. For example, the monitor 102 (of FIG. 1) could include more than two electrodes such that multiple sets of time-synchronized ECG data are recorded. One combination of electrodes can sense ECG data for a first channel, a second combination of electrodes can sense ECG data for a second channel, and a third combination of electrodes can sense ECG data for a third channel—all of which are time synchronized over a strip of time.

FIG. 6 shows an index 350 structure such that individual compressed ECG data blobs (and therefore their compressed ECG data blocks) can be identified and selected. The index 350 includes the start time 352 for each compressed ECG data blob, the end time 354 for each compressed ECG data blob, a blob block offset value 356 for each compressed ECG data blob, and a blob block size 358 for each compressed ECG data blob. As noted above, each compressed ECG data blob can contain a contiguous set of ECG data, so the start time 352 and the end time 354 can represent the beginning time and ending time of that contiguous set of ECG data. Each compressed ECG data blob can be listed in a row in the index 350, and the index 350 can have an N-number of rows 360A-N depending on the number of compressed ECG data blobs within a given study.

Using the index 350, individual compressed ECG data blobs can be selected (e.g., using a pointer). The compressed ECG data blobs allow for strips of compressed ECG data to be compiled into more manageable larger data structures instead of having to reference potentially thousands of individual strips of ECG data.

Referring back to FIG. 1 and FIG. 3, the method 200 includes transmitting the compressed packages to the remote computer 116 (block 206 in FIG. 3).

In certain instances, the process of transmitting the compressed ECG data from the server 106 to the remote computer 116 starts when a user at the remote computer 116 selects a patient or study for analysis. For example, the server 106 can host a SaaS platform that allows a user to review a list of patients or studies ready for analysis on the remote computer 116. Once a patient or study is selected, the remote computer 116 can begin transmitting commands to the server 106 for packages of data or the server 106 can begin pushing/transmitting packages of data to the remote computer 116.

One package of data can include executable code (e.g., JavaScript code, C code) that can be executed on a browser operated on the remote computer 116. For example, the browser can utilize WebAssembly (sometimes referred to as WASM) to run executable code on the browser. The executable code can include one or more modules for decompressing the compressed ECG data once the compressed ECG data has been transmitted to and stored at the remote computer 116.

In addition to the executable code, the server 106 can transmit to the remote computer 116 metadata associated with the ECG data. The metadata may include cardiac event classifications and associated indicators (e.g., start time, end time, compressed ECG data blob number) that identify where a given cardiac event occurred within the study. Other metadata can include heart rates (e.g., maximum heart rates), R-R intervals (e.g., longest RR intervals), and other data that characterizes the underlying ECG data. In certain instances, the metadata is not compressed.

In addition to the executable code and metadata, the server 106 can begin transmitting the compressed packages in collective groups (e.g., compressed ECG data blobs). In certain instances, the compressed packages of ECG data (individually or in collective groups) begin being transmitted based on an initial priority. For example, in certain instances, the ECG data associated with highest priority cardiac events are initially transferred. The highest priority cardiac events can be the cardiac events classified as being the most critical. And if a given compressed ECG data blob contains the time period of ECG data during which such cardiac events occurred, the entire compressed ECG data blob can be transmitted to the remote computer 116 on a priority basis. In certain instances, the remote computer 116 uses the transferred metadata to determine which ECG data should be requested from the server 106. In other instances, the server 106 pushes/transmits the priority ECG data to the remote computer 116.

After the initial packages of data (e.g., executable code, metadata, and ECG data containing the highest priority cardiac events) are transmitted from the server 106 to the remote computer 116, additional packages of data can be transmitted in response to selections made by the user in the UI 122. For example, if a user uses the UI 122 to select a cardiac event or scroll towards a portion of the study not yet transmitted, the remote computer 116 can request the relevant compressed ECG data from the server 106.

In certain instances, compressed packages of ECG data can be downloaded in parallel. For example, the browser on the remote computer 116 can employ multi-threading techniques such that separate packages of compressed ECG can be downloaded simultaneously.

Once a given compressed package of ECG data is downloaded to the remote computer 116, the remote computer 116 can decompress the ECG data into strips of ECG data. In certain instances, the executable code transmitted from the server 106 to the remote computer 116 can include code to operate a decompression process. Because of the compression approaches described above, the decompression process consumes fewer computing resources (e.g., power, calculations) compared to traditional decompression processes. In certain instances, the compressed packages of ECG data that are downloaded to the remote computer 116 first are also decompressed first such that the ECG data can be displayed on the UI 122 while other compressed packages of ECG data are being downloaded.

The decompression process can be carried out by performing inverse operations to that shown in FIG. 4. For example, an unpacking algorithm can be applied to the compressed ECG data such that the bit packing is reversed. Once the bit packing is reversed, the resulting data can be remapped (or converted back) to their initial positive values and initial negative values. If Equation 2 above was used for the initial conversion, Equation 3 below can be used to convert the values back to their initial positive values and initial negative values (e.g., by decoding the prior encoded ECG data):

$${z}_{i} = ( {d}_{i} >>> 1 ) ⊕ - ( {d}_{i} \& 1 ) \qquad Equation 3$$

Once the ECG data is converted back to the initial positive values and initial negative values, the bias previously reduced can be reintroduced or added back to the ECG data. If Equation 1 above was used for the initial conversion, Equation 4 below can be used to reintroduce bias (e.g., by decoding the prior encoded ECG data):

$${e}_{i} = {d}_{i} + {e}_{i - 1} \qquad Equation 4$$

The compression approaches described above can be considered to be lossless compression approaches (as opposed to lossy compression approaches). When uncompressed, the ECG data is reconstructed to its original form with little to no lost data.

The uncompressed ECG data can then be displayed on the UI 122 on the remote computer 116 to facilitate analysis of the patient's cardiac activity.

Although the examples described above are focused on compressing ECG data, other types of time-series data could be compressed (and decompressed) using techniques such as preconditioning time-series data and compressing the preconditioned time-series data (e.g., by using bit packing approaches).

### Computing Devices and Systems

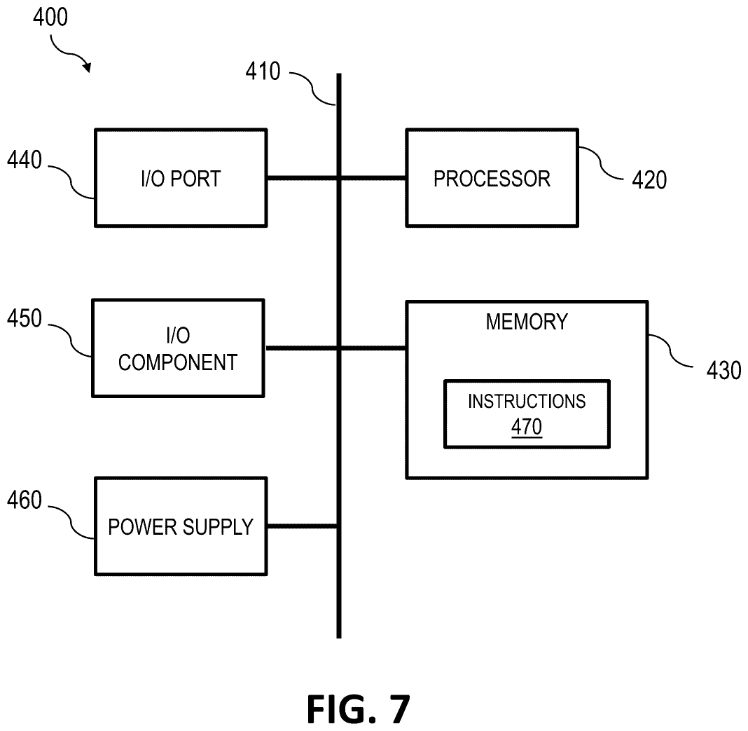

FIG. 7 is a block diagram depicting an illustrative computing device 400, in accordance with instances of the disclosure. The computing device 400 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 7 and described below. For example, the monitor 102, the mobile device 104, the server 106, and the remote computer 116 can each include their own set of components shown in FIG. 7 and described below.

In instances, the computing device 400 includes a bus 410 that, directly and/or indirectly, couples one or more of the following devices: a processor 420, a memory 430, an input/output (I/O) port 440, an I/O component 450, and a power supply 460. Any number of additional components, different components, and/or combinations of components may also be included in the computing device 400.

The bus 410 represents what may be one or more busses (such as, for example, an address bus, data bus, or combination thereof). Similarly, in instances, the computing device 400 may include a number of processors 420, a number of memory components 430, a number of I/O ports 440, a number of I/O components 450, and/or a number of power supplies 460. Additionally, any number of these components, or combinations thereof, may be distributed and/or duplicated across a number of computing devices.

In instances, the memory 430 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 430 stores computer-executable instructions 470 for causing the processor 420 to implement aspects of instances of components discussed herein and/or to perform aspects of instances of methods and procedures discussed herein. The memory 430 can comprise a non-transitory computer readable medium storing the computer-executable instructions 470.

The computer-executable instructions 470 may include, for example, computer code, machine-useable instructions, and the like such as, for example, program components capable of being executed by one or more processors 420 (e.g., microprocessors) associated with the computing device 400. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 470 may be configured to be executed by the processor 420 and, upon execution, to cause the processor 420 to perform certain processes. In certain instances, the processor 420, memory 430, and instructions 470 are part of a controller such as an application specific integrated circuit (ASIC), field-programmable gate array (FPGA), and/or the like. Such devices can be used to carry out the functions and steps described herein.

The I/O component 450 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a server comprising: one or more processors, and computer-readable media having computer-executable instructions embodied thereon, the instructions configured to be executed by the one or more processors to cause the server to:
process strips of electrocardiogram (ECG) data to generate compressed packages of the ECG data, wherein the process includes:
generating preconditioned ECG data, and
compressing the preconditioned ECG data; and
transmit the compressed packages of the ECG data to a remote computing system.

2. The system of claim 1, wherein the generating the preconditioned ECG data includes processing the strips of ECG data using a first encoding process to generate a first set of encoded ECG data, and then processing the first set of encoded ECG data to generate a second set of encoded ECG data, wherein the second set of encoded data is the preconditioned ECG data.

3. The system of claim 1, wherein the generating the preconditioned ECG data includes reducing bias in the ECG data, wherein the compressing the preconditioned ECG data includes using bit packing.

4. The system of claim 3, wherein, after the reducing the bias, the ECG data contains initial negative values and initial positive values.

5. The system of claim 4, wherein the generating the preconditioned ECG data further includes: converting the initial negative values to subsequent positive values.

6. The system of claim 5, wherein the subsequent positive values are interleaved with the initial positive values to generate the preconditioned ECG data.

7. The system of claim 3, wherein the reducing the bias in the ECG data includes applying a differential encoding algorithm to the ECG data.

8. The system of claim 1, wherein the server comprises a machine learning mode, wherein the instructions are configured to be executed by the one or more processors to cause the server to:
before the strips of the ECG data are processed to generate the compressed packages of the ECG data, input the strips of the ECG data into the machine learning model to generate metadata, the metadata including cardiac event classifications and beat classifications.

9. The system of claim 1, wherein the compressed packages of the ECG data include a collection of separate blocks of compressed strips of ECG data.

10. The system of claim 1, wherein the compressed packages of the ECG data that contain a critical cardiac event are transmitted to the remote computing system before the compressed packages of ECG data that do not contain a critical cardiac event.

11. The system of claim 1, wherein the instructions are configured to be executed by the one or more processors to cause the server to:
transmit executable code to the remote computing system, wherein the executable code is configured to be executed by the remote computing system to decompress the compressed packages of the ECG data.

12. The system of claim 11, further comprising:
the remote computing system comprising a set of its own one or more processors arranged to execute the executable code to cause the remote computing system to:
decompress the compressed packages of the ECG data into the preconditioned ECG data, and
decode the preconditioned ECG data to reconstruct the strips of the ECG data.

13. The system of claim 12, wherein the remote computing system includes a user interface, wherein the user interface is configured to display the strips ECG data after being decompressed and decoded.

14. The system of claim 12, wherein the remote computing system is configured to operate a browser, wherein the browser is configured to execute the executable code received from the server.

15. A method comprising:
receiving one or more electronic files that contain strips of time-series data;
processing the strips of the time-series data to generate compressed packages of the time-series data, wherein the processing includes:
generating preconditioned time-series data by a first encoding process that converts the time-series data to a first set of encoded time-series data and then a second encoding process that converts the first set of encoded time-series data to the preconditioned time-series data, and
compressing the preconditioned time-series data; and
transmitting the compressed packages of the time-series data to a remote computing system.

16. The method of claim 15, wherein the first set of encoded time-series data includes initial negative values and initial positive values.

17. The method of claim 16, wherein the second set of encoded time-series data only includes positive values.

18. The method of claim 17, wherein the positive values are compressed using bit packing.

19. The method of claim 17, wherein the positive values include subsequent positive values converted from the initial negative values, wherein the subsequent positive values are interleaved with the initial positive values to generate the preconditioned time-series data.

20. The method of claim 15, wherein the first encoding process applies a differential encoding algorithm to the time-series data.
